(19) United States
(12) Patent Application Publication
(10) Pub. No.: US 2006/0089559 A1
(43) Pub. Date: Apr. 27, 2006

(54) Realtime monitoring and analysis of heart-beat dynamics

(76) Inventors: Riccardo Barbieri, Boston, MA (US); Eric C. Matten, Brookline, MA (US); Emery N. Brown, Brookline, MA (US)

Correspondence Address: FISH & RICHARDSON PC, P.O. BOX 1022, MINNEAPOLIS, MN, 55440-1022, US

(21) Appl. No.: 11/209,195

(22) Filed: Aug. 22, 2005

Related U.S. Application Data
(60) Provisional application No. 60/604,755, filed on Aug. 26, 2004.

(51) Int. Cl. A61B 5/0402

(52) U.S. Cl. 600/509

(57) ABSTRACT

A system for estimating a statistic associated with a cardiac signal having periodic events includes a clock and a digital filter. The clock determines an extent of a censored interval beginning at a most recent event and ending at a time selected from a continuum of times. The digital filter implements a point process stochastic model of the cardiac signal, receives the censored interval, and estimates the statistic at least in part on the basis of historical data and the censored interval.

### RELATED APPLICATIONS

This application claims priority under 35 U.S.C. §119 to U.S. Provisional Patent Application Ser. No. 60/604,755, filed Aug. 26, 2004, the entire contents of which are hereby incorporated by reference.

### FIELD OF INVENTION

This invention is related to processing of physiological signals, and in particular, to the processing of periodic signals whose periods fluctuate.

### BACKGROUND

Certain processes within the human body are periodic in nature. One example of such a process is the beating of the heart. However, although the heart beats periodically, the heart rate fluctuates. For example, when one sleeps, one's heart beats slowly. When one sprints, one's heart beats more quickly.

A common method for determining a heart rate is to count the number of heart beats that occur during some measurement interval. However, this does not result in a value of the heart rate at a particular instant. Instead, this provides an estimate of the heart rate at that instant on the basis of an average value of the heart rate during the course of a measurement interval that preceded that instant.

Moreover, the averaging process is fundamentally flawed because heart rate is not constant. As a result, one cannot arbitrarily improve the accuracy of the estimate by simply taking measurements over a longer interval.

In many cases, both the heart rate and the heart rate variability are of interest. For example, if one's heart were still racing some time after having sprinted some distance, one would know instinctively that something was wrong. In the context of fetal monitoring, the interplay between the fetal heart rate variability and the mother's contractions can be crucial to determining whether a C-section should begin immediately. Loss of heart-rate variability is also useful as an indicator of ventricular dysfunction in patients with congestive heart failure, and as an independent predictor of mortality following acute myocardial infarction. Heart rate variability is also of interest in the diagnosis of diseases of the autonomic nervous system, such as diabetes, Guillain-Barre syndrome, multiple sclerosis, Parkinson's disease, and Shy-Drager orthostatic hypertension.

The method by which heart rate variability is now determined belies its diagnostic importance. Typically, one simply makes a subjective judgment about the heart rate variability upon visual inspection of a trace of the heart rate.

### SUMMARY OF THE INVENTION

The invention provides a method and system for providing an estimate of instantaneous values of statistics (e.g. mean, variance, kurtosis, etc.) associated with a heart beat. The method and systems do so by modeling the periodic heartbeat as a dynamic point process.

In one aspect, the invention includes a method for estimating a statistic associated with a cardiac signal having periodic events. In this method, a point process stochastic model of the cardiac signal is defined, and a selection is made of, from a continuum of times, of a selected time at which to estimate the statistic. An extent of a censored interval is determined. On the basis of the point process model and the extent of the censored interval, an estimate of the statistic at the selected time is computed.

Practices of the invention include those in which the stochastic model is selected to have a history-dependent inverse Gaussian probability density function.

Other practices include those in which a parameter associated with the stochastic model is selected at least in part on the basis of a history of intervals. The parameter can be estimated, for example, by computing a maximum likelihood estimate and by recursively updating the parameter estimate.

Another aspect of the invention includes a computer-readable medium having encoded thereon software for causing a computer to execute any of the foregoing methods.

In another aspect, the invention includes a system for estimating a statistic associated with a cardiac signal having periodic events. The many embodiments of such a system include a clock for determining an extent of a censored interval beginning at a most recent event and ending at a time selected from a continuum of times, as well as a digital filter for implementing a point process stochastic model of the cardiac signal and receiving the censored interval. The digital filter is configured to estimate the statistic at least in part on the basis of historical data and the censored interval.

Among the embodiments are those that also include a signal source for providing the cardiac signal. Such a signal source can be, for example, an electrocardiograph, or a fetal heart monitor. However, the invention can be used with any of a variety of periodic or quasi-periodic signals.

Also among the embodiments are those in which the digital filter is configured to implement a stochastic model having a history dependent inverse Gaussian probability density function. However, the digital filter can be configured to implement any stochastic model, with the particular choice of model being selected on the basis of the underlying signal.

### BRIEF DISCRIPTION OF FIGURES

FIG. 1 is a schematic illustration of a system for estimating statistics in real-time.

FIG. 2 is a graph of measured R-R intervals from four test subjects.

FIG. 3 is a graph comparing probabilistic models for the measurements shown in FIG. 2.

FIG. 4 shows scatter plots that compare the independence of R-R intervals for each of the models compared in FIG. 3.

FIG. 5 is a graph that compares heart rates determined by each of the models shown in FIG. 3 during a tilt-table study.

FIG. 6 compares heart-rate variability and R-R interval variability as derived from the same heart rate signal.

FIG. 7 shows box-plot summaries corresponding to the data shown in FIG. 2.

### DETAILED DESCRIPTION

The methods and systems described herein provide a digital filter that generates real-time statistics for a variety of periodic physiological signals, such as heart beats.

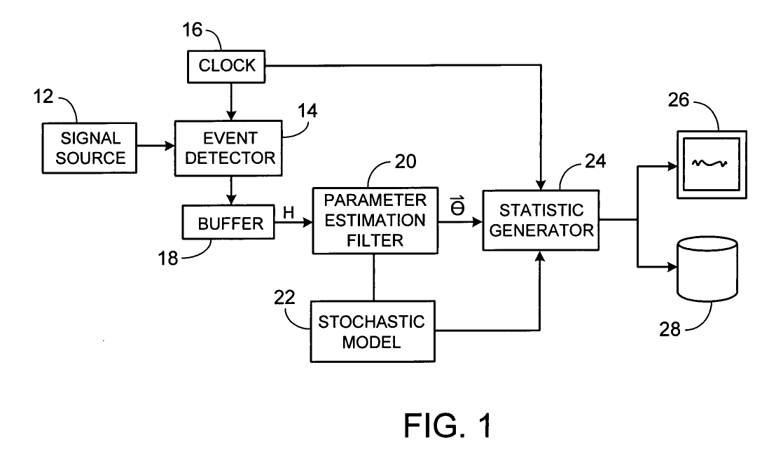

Referring to FIG. 1, a system 10 for carrying out the methods described herein includes a signal source 12 for measuring a periodic physiological signal. The output of the signal source 12 is a sequence of events.

These events are provided to an event detector 14 connected to a clock 16. The event detector 14 uses the clock 16 to tag each event with a time of occurrence. As each event occurs, the event detector 14 determines an interval between that event and an immediately preceding event. The resulting sequence of intervals is then provided to a buffer 18. The buffer 18 stores the most recent interval and a selected number of intervals immediately preceding the most recent interval. To accommodate the most recent interval, the buffer 18 discards the oldest interval. In so doing, the buffer 18 maintains a record of recent intervals.

The intervals in the buffer 18 are provided to a parameter-estimation filter 20 that maintains a history vector. The elements of the history vector include intervals stored in the buffer 18 and one additional element, namely the time at which the most recent event occurred. The length of the interval that begins with the most recently occurring event cannot be determined until another event occurs to end it, e.g. until the heart beats once again. The time of the most recent event thus determines the beginning of an interval whose endpoint is not yet known. Such an interval is referred to as a “censored” interval, or, in recognition of the manner in which time is customarily depicted, a “right-censored” interval.

The extent, or duration of this censored interval changes continuously with time. This extent contains information that can be used to adjust estimates of interval statistics based only on completed intervals. An estimate adjusted in the manner described herein thus amounts to a real-time estimate of the average interval at any instant, and in particular, at those instants that fall between events.

The parameter-estimation filter 20 has a filter transfer function that implements a pre-determined stochastic model 22 of the periodic physiological signal. The choice of stochastic model 22 depends on the underlying physiology that resulted in the signal. For example, in the case of an electrocardiogram, an appropriate stochastic model 22 is one that models a point process having a history-dependent inverse Gaussian probability density function.

In most cases, the stochastic model 22 will have certain parameters derivable from the history vector. These parameters are estimated by the parameter-estimation filter 20 on the basis of the history vector.

The particular method used to estimate the parameters depends in part on the nature of the stochastic model 22, which, as noted above, itself depends on the underlying physiological processes that resulted in the signal. Where the underlying process is the heart beat, the parameter-estimation filter 20 uses a maximum likelihood function to estimate the parameters. However, the parameter-estimation filter 20 can also be a recursive filter that maintains a previous estimate of the parameter and continually updates that estimate on the basis of a current duration of the censored interval.

The output of the parameter-estimation filter 20, which includes the estimate of the parameters associated with the stochastic model 22, is then provided to a statistic generator 24. The statistic generator 24 recognizes the stochastic model 22, which is a continuous function of time. It also receives a parameter estimate from the parameter-estimation filter 20. Additionally, the statistic generator 24 receives the current time from the clock 16. As a result, the statistic generator 24 has all that it needs to evaluate, at any instant, a value of the stochastic model 22 that is consistent with the history vector.

The output of the statistic generator 24 can assume several forms, all of which are derivable from the stochastic model 22. For example, the output can be the average interval between events as a function of time, or the reciprocal thereof. Higher order moments, such as the time-varying variance or kurtosis associated with intervals between events, or reciprocals thereof, can also be provided.

The output(s) of the statistic generator 24 are then provided to a display unit 26 or stored on a computer-readable medium 28 for further analysis.

While the foregoing methods can be applied to the analysis of any periodic physiological signal, the remainder of this description is of a particular embodiment in which the periodic physiological signal is derived from cardiac activity. In this case, the signal source 12 is an electrocardiogram (“ECG”).

A method for determining instantaneous heart rate values begins with obtaining, from an ECG, K successive R-wave event times 0<u₁<u₂<, . . . ,<uₖ<, . . . ,<u_{K}≦T during an observation interval (0, T). The interval between two successive R_wave events will be referred to as an R-R interval.

An inverse Gaussian distribution is like a Gaussian but skewed such that its peak shifts toward the origin. The resulting distribution is thus asymmetric about its mean. The rapid drop-off in the inverse Gaussian distribution as one approaches the origin is consistent with the existence of a refractory interval between muscle contractions. The slower drop-off as one proceeds toward infinity is consistent with the greater variation in heart beat interval as the heart slows down following the cessation of cardiovascular exertion.

The probability density of the R-R intervals is assumed to be inverse Gaussian. The series of R-R intervals can thus be modeled as inverse Gaussian random variables. Because the effects of sympathetic and/or parasympathetic inputs to the SA node persist, R-R interval lengths are preferably modeled by a function of the history of inputs to the SA node. Furthermore, because the sympathetic and parasympathetic inputs to the SA node are part of the cardiovascular control circuitry, these inputs are dynamic. By considering the effect of previous autonomic inputs to the SA node, the method described herein accommodates the dynamic character of these inputs.

The processing method described herein assumes that the length of an R-R interval is described by a history-dependent inverse Gaussian (“HDIG”) probability density function:

$$f ( t \vert {H}_{{u}_{k}} , θ ) = {[ \frac{{θ}_{p + 1}}{2 {π ( t - {u}_{k} )}^{3}} ]}^{\frac{1}{2}} exp \{ - \frac{1}{2} \frac{{{θ}_{p + 1} [ t - {u}_{k} - μ ( {H}_{{u}_{k}} , θ ) ]}^{2}}{{μ ( {H}_{{u}_{k}} , θ )}^{2} ( t - {u}_{k} )} \} \qquad [ 1 ]$$

where uₖ is the time at which the most recent event occurred. The variable t is the time at which an estimate is sought. The constant θ is one element of a parameter vector \vec{θ} associated with the model. The function μ is a function that returns a mean when provided with a history of recent values Hᵤ_{ₖ} of the random variable being modeled. The foregoing model thus represents the dependence of the R-R interval lengths on the recent history of parasympathetic and sympathetic inputs to the SA node. It does so by modeling the mean at any instant t as a linear function of the lengths of the preceding p R-R intervals. If we assume that the R-R intervals are independent (i.e., by assuming p=0), then μ(Hᵤ_{ₖ}, θ)=θ₀, and ƒ(t|Hᵤ_{ₖ}, θ)=ƒ(t|uₖ, θ₀, θ₁). In this case, the probability density function of equation (1) simplifies to a renewal inverse Gaussian (“RIG”) model. The mean, μ_{RR}, and standard deviation σ_{RR}, of the R-R probability model exemplified by equation (1) are respectively,

$$\begin{matrix}{μ}_{RR} = μ ( {H}_{{u}_{k}} , θ ) & [ 2 ] \\ {σ}_{RR} = {[ {μ ( {H}_{{u}_{k}} , θ )}^{3} {θ}_{p + 1}^{- 1} ]}^{\frac{1}{2}} & [ 3 ]\end{matrix}$$

Since the probability density function of equation (1) characterizes the stochastic properties of the R-R interval lengths, it can be used to formulate precise definitions of heart rate (which is the reciprocal of the R-R interval lengths) and heart rate variability. For t>uₖ, the difference t−uₖ is the waiting time until the next R-wave event. The quantity r=c(^{t-u}^{ₖ})⁻¹ is the heart rate, where c is a constant that converts interval measurements into heart rate measurements. Since r is a one-to-one transformation of t−uₖ, the heart rate probability density can be derived from the interval probability density in equation (1) as:

$$\begin{matrix}f ( r \vert {H}_{{u}_{k}} , θ ) = \vert \frac{ⅆ t}{ⅆ r} \vert f ( t \vert {H}_{{u}_{k}} , θ ) \\ = {[ \frac{{θ}_{p + 1}^{*}}{2 π r} ]}^{\frac{1}{2}} exp \{ - \frac{1}{2} \frac{{{θ}_{p + 1}^{*} [ 1 - {μ}^{*} ( {H}_{{u}_{k}} , θ ) r ]}^{2}}{{{μ}^{*} ( {H}_{{u}_{k}} , θ )}^{2} r} \} ,\end{matrix} \qquad [ 4 ]$$

where μ(Hᵤ_{ₖ}, θ)=c⁻¹μ(Hᵤ_{ₖ}, θ) and θ*_{p+1=c}⁻¹ θₚ₊₁. The mean (μ_{HR}), mode (\tilde{μ}_{HR}), and standard deviation (σ_{HR}) of the heart rate probability density are respectively

$$\begin{matrix}{μ}_{HR} = {{μ}^{*} ( {H}_{{u}_{k}} , θ )}^{- 1} + {θ}_{p + 1}^{* - 1} & [ 5 ] \\ {\tilde{μ}}_{HR} = {{μ}^{*} ( {H}_{{u}_{k}} , θ )}^{- 1} [ {( 1 + \frac{{{μ}^{*} ( {H}_{{u}_{k}} , θ )}^{2}}{4 {θ}_{p + 1}^{* 2}} )}^{\frac{1}{2}} - \frac{{μ}^{*} ( {H}_{{u}_{k}} , θ )}{2 {θ}_{p + 1}^{*}} ] & [ 6 ] \\ {σ}_{HR} = {[ \frac{2 {μ}^{*} ( {H}_{{u}_{k}} , θ ) + {θ}_{p + 1}^{*}}{{μ}^{*} ( {H}_{{u}_{k}} , θ ) \cdot {θ}_{p + 1}^{* 2}} ]}^{\frac{1}{2}} & [ 7 ]\end{matrix}$$

Equation (4) provides a model of the stochastic properties of heart rate in terms of a probability density. If p=0, then Equation (4) gives the simple reciprocal of the inverse Gaussian probability density. To use equation (4) in analyses of R-R interval time-series, heart rate should be a representative value from the heart rate probability density. Therefore, heart rate is defined as either the mean, which is shown in equation (5), or the mode, which is shown in equation (6), of ƒ(r|Hᵤ_{ₖ}, θ); the heart rate variance is defined as the square of the standard deviation, as shown in equation (7).

The function ƒ(t|Hᵤ_{ₖ}, θ), shown in equation (1) is thus a probability model for the R-R intervals. It represents the effects of recent sympathetic and parasympathetic input to the SA node through its mean parameter, μ(Hᵤ_{ₖ}, θ). A standard change-of-variables transforms the R-R interval probability density function of equation (1) into a heart rate probability density function, ƒ(r|Hᵤ_{ₖ}, θ), as shown in equation (4). A time-varying vector of model parameters, θ, accounts for the continuous dynamics of the sympathetic and parasympathetic inputs to the SA node. Hence, by tracking the time-course of θ, and continuously evaluating equations (5) and (7) as θ evolves, it is possible to track instantaneous heart rate and heart rate variability respectively.

A local maximum-likelihood procedure estimates the time-varying parameter θ for use in equations (5) and (7). For a semi-open ECG recording interval (0,T], let l be the length of the local likelihood observation interval, and let Δbe the shift parameter. The shift parameter defines an extent to which the local likelihood time interval is shifted to compute the next parameter update. Let t^{l} be the local likelihood observation interval (t−l,t] for t∈[l,T], and assume that, within the interval t^{l}, there are nₜ observed R-wave event times: t−l<u₁<u₂< . . . <uₙ_{ₗ}≦t. Let u_{t-1:t}={u₁, . . . , uₙ_{ₗ}} be the sequence of R-wave event times. The local maximum likelihood estimate of θ at time t, namely \hat{θ}ₜ, is obtained by first defining the local joint probability density of the R wave event times in the sequence u_{t-l:t}. If one conditions on the p R-R intervals preceding each R-wave event in the R-wave event sequence u_{t-l:t}, then the (nₜ+1) interval of the local likelihood observation interval t^{l} is not completely observed. This results in right-censoring of the R-R interval measurements. Taking account of this right-censoring, the local-log likelihood is:

$$log f ( {u}_{t - l : t} \vert {θ}_{t} ) = {\sum }_{i = 2}^{{n}_{t}} w ( t - {u}_{i} ) log f ( {u}_{i} - {u}_{i - 1} \vert {H}_{{u}_{i - 1}} , {θ}_{t} ) + w ( t - {u}_{{n}_{t}} ) log {\int }_{t - {u}_{{n}_{t}}}^{∞} f ( v \vert {H}_{{u}_{{n}_{t}}} , {θ}_{t} ) ⅆ v . \qquad [ 10 ]$$

where the first term corresponds to the completed intervals and the second term corresponds to the right-censored interval.

A suitable procedure for computing a value of \hat{θ}ₜ to maximize the local log likelihood shown in equation (10) is the Newton-Raphson procedure. Because of potentially significant overlap between adjacent local likelihood intervals, the Newton-Raphson procedure at t starts with the previous local maximum likelihood estimate at time t−Δ. Once \hat{θ}ₜ is computed, the interval t^{l} is shifted to (t−l+Δ,t+Δ] and the local maximum likelihood estimation is repeated. This procedure is continued until t=T.

Given \hat{θ}ₜ, the local maximum likelihood estimate of θat time t, it follows from equations (5) and (7) that the instantaneous estimates of the mean R-R interval length (μ_{RR}), the R-R interval standard deviation (σ_{RR}_{ₜ}), the mean heart rate (\tilde{μ}_{HRt}), and the heart rate standard deviation at time t (σ_{HR}_{ₜ}) are respectively:

$$\begin{matrix}{μ}_{{RR}_{t}} = μ ( {H}_{t} , {θ}_{t} ) & [ 11 ] \\ {σ}_{{RR}_{t}} = {( {μ ( {H}_{t} , {θ}_{t} )}^{3} {θ}_{p + 1 , t}^{- 1} )}^{\frac{1}{2}} & [ 12 ] \\ {\hat{μ}}_{{HR}_{t}} = {{μ}_{t}^{*} ( {H}_{t} , {\hat{θ}}_{t} )}^{- 1} + {\hat{θ}}_{p + 1 , t}^{* - 1} & [ 13 ] \\ {\hat{σ}}_{{HR}_{t}} = {[ \frac{2 {μ}_{t}^{*} ( {H}_{t} , {\hat{θ}}_{t} ) + {\hat{θ}}_{p + 1 , t}^{*}}{{μ}_{t}^{*} ( {H}_{t} , {\hat{θ}}_{t} ) \cdot {\hat{θ}}_{p + 1 , t}^{* 2}} ]}^{\frac{1}{2}} . & [ 14 ]\end{matrix}$$

Another method for determining the parameters θ associated with the probability model is to use a point-process adaptive filter to recursively update the parameter. In this method, one divides the ECG recording interval (0, T] into K sub-intervals of equal width Δ=T/K. The number of such sub-intervals, K, is selected to be large enough to ensure that there is no more than one event in each sub-interval. The method further assumes that the temporal dynamics of the parameters θ are given by:
θₖ=θₖ₋₁+εₖ [15]
where εₖ is zero-mean Gaussian noice with variance W_{ε}.

The use of an adaptive filter includes defining a state-space representation of the particular stochastic process being modeled, which in this case is the point-process having a history-dependent inverse Gaussian distribution as defined by equation (1).

The state-space representation includes a state process model and an observation process model. In this case, the state process model is an associated conditional density function obtained from equation (1):

$$λ ( k Δ \vert {H}_{k} , {θ}_{k} ) = \frac{f ( k Δ \vert {H}_{k} , {θ}_{k Δ} )}{1 - {\int }_{{u}_{k}}^{k Δ} f ( u \vert {H}_{k} , {θ}_{u} ) ⅆ u} , \qquad [ 16 ]$$

For a small sub-interval width Δ, equation (16) represents the probability that an event will occur in the interval [(k−1)Δ, kΔ].

The observation model is a representation of a probability mass function in terms of the conditional intensity function:
Pr(dN(kΔ)|Hₖ, θₖ)=exp {dN(kΔ)log [λ(kΔ|Hₖ, θ_{kΔ})Δ]−λ(kΔ|Hₖ,θ_{kΔ})Δ}. [17]

Given the foregoing process model and observation model, the corresponding adaptive filter algorithm is given by:
θ_{k|k-1}=k−1|k−1 [18]
W_{k|k-1}=W_{k-1|k-1}+W_{ε} [19]
θ_{k|k}=θ_{k|k-1}+W_{k|k-1}(∇ log λₖ)[dN(kΔ)−λₖΔ] [20]

$${W}_{k \vert k} = - {[ - {W}_{k \vert k - 1} + ( {∇}^{2} log {λ}_{k} ) [ d N ( k Δ ) - {λ}_{k} Δ ] - {( ∇ log {λ}_{k} ) [ ∇ {λ}_{k} Δ ]}^{′} ]}^{- 1} \qquad [ 21 ]$$

where equation (18) is the one-step prediction, equation (19) is the one-step prediction variance, equation (20) is the posterior mode equation, and equation (21) is the posterior variance. In equations (20) and (21), λₖ=λ(kΔ|Hᵤ_{ₖ}, θ_{k|k-1}), ∇ denotes the first derivative of the indicated function with respect to θ, and ∇² denotes the second derivative of the indicated function with respect to θ.

Given \hat{θ}ₖ, which is the point-process adaptive filter estimate for θ at time kΔ, it follows from equations (2), (3), (6), and (7), that the instantaneous estimates at time kΔ for the mean and standard deviation of the R-R interval are

$$\begin{matrix}{μ}_{RR} ( k Δ ) = μ ( {H}_{k} , {\hat{θ}}_{k} ) & [ 15 ] \\ {σ}_{RR} ( k Δ ) = {[ {μ ( {H}_{k} , {\hat{θ}}_{k} )}^{3} {\hat{θ}}_{p + 1 , k}^{- 1} ]}^{\frac{1}{2}} . & [ 16 ]\end{matrix}$$

and that the instantaneous estimates at time kΔfor the mean and standard deviation of the heart rate are

$$\begin{matrix}{μ}_{HR} ( k Δ ) = {{μ}^{*} ( {H}_{k} , {\hat{θ}}_{k} )}^{- 1} + {\hat{θ}}_{p + 1 , k}^{* - 1} & [ 17 ] \\ {σ}_{HR} ( k Δ ) = {[ \frac{2 {μ}^{*} ( {H}_{k} , {\hat{θ}}_{k} ) + {\hat{θ}}_{p + 1 , k}^{*}}{{μ}^{*} ( {H}_{k} , {\hat{θ}}_{k} ) \cdot {\hat{θ}}_{p + 1 , k}^{* 2}} ]}^{\frac{1}{2}} & [ 18 ]\end{matrix}$$

The foregoing equations thus provide continuous time estimates of statistics associated with heart rate and heart rate variability. This is because the history-dependent inverse Gaussian model, and hence the heart rate probability model, is defined in continuous time. For any t∈(l, T], the local likelihood procedure uses all of the data in the local likelihood interval (t−l,t] to compute \hat{θ}ₜ. If the observation time t coincides with an R-event, (t=uₙ_{ₜ}), the contribution to the local likelihood is

$$log f ( {u}_{{n}_{t}} - {u}_{{n}_{t}} - 1 \vert {H}_{{u}_{{n}_{t}}} , θ ) .$$

If, on the other hand, the observation time follows an R-event, (t>uₙ_{ₜ}) then the last interval is right-censored and the contribution to the local likelihood becomes

$$log {\int }_{t - {u}_{{n}_{t}}}^{∞} f ( v \vert {H}_{{u}_{{n}_{t}}} , {θ}_{t} ) ⅆ v .$$

Thus, regardless of whether it is completely observed or censored, the last interval contributes to the estimate. This means that the local likelihood function is defined at t, which is both the right endpoint of the observation interval and the point at which the local maximum likelihood estimate is to be computed. Since θₜ can be estimated in continuous time, heart rate and heart rate variability, both of which are continuous functions of θₜ, can also be estimated in continuous time. The R-R interval probability model, together with the local maximum likelihood method of estimating dynamically varying model parameters, provides an approach for estimating the instantaneous mean R-R interval length, the mean heart rate, the R-R interval standard deviation, and the heart rate standard deviation from a time-series of R-R intervals. For this approach, it is also useful to determine how well the model describes the series of R-R intervals. Because equation (1) defines an explicit point process model, the time-rescaling theorem can be used to evaluate model goodness-of-fit. To do so, time-rescaled, or transformed, R-R intervals are first defined by

$${τ}_{k} = {\int }_{{u}_{k - 1}}^{{u}_{k}} λ ( t \vert {H}_{{u}_{{n}_{t}}} , {\hat{θ}}_{t} ) ⅆ t \qquad [ 15 ]$$

where the R-wave event times uₖ define a point process observed in the semi-open ECG observation interval (0,T], and

$$λ ( t \vert {H}_{{u}_{{n}_{t}}} , {\hat{θ}}_{t} )$$

is the conditional intensity

$$λ ( t \vert {H}_{{u}_{{n}_{t}}} , {\hat{θ}}_{t} ) = {f ( t \vert {H}_{{u}_{{n}_{t}}} , {\hat{θ}}_{t} ) [ 1 - {\int }_{{u}_{{n}_{t}}}^{t} f ( v \vert {H}_{{u}_{{n}_{t}}} , {\hat{θ}}_{t} ) ⅆ v ]}^{- 1} \qquad [ 16 ]$$

evaluated at the local maximum likelihood estimate \hat{θ}ₜ. For a point process, the conditional intensity is a history-dependent rate function that generalizes the rate function for a Poisson process. On the basis of the time-rescaling theorem, the τₖ are independent, exponential random variables with a unit rate. Under the further transformation zₖ=1−exp(−τₖ), the zₖ are independent, uniform random variables on the semi-open interval (0,1]. Therefore, we can construct a Kolmogorov-Smimov (“KS”) test to assess agreement between the zₖ and the uniform probability density. Because the transformation from the uₖ to the zₖ is one-to-one, there will be close agreement between the zₖ and the uniform probability density if and only if there is also close agreement between the point process probability model and the series of R-R intervals. Hence, the time-rescaling theorem provides a way to measure agreement between a point process probability model and an R-R interval series.

A local maximum likelihood analysis begins by setting p, the order of the history dependence in Equation (1) and l, the length of the local likelihood interval. These two parameters are chosen in part on the basis of the approximate Akaike's Information Criterion (“AIC”) for local maximum likelihood analyses.

The HDIG model described herein provides an explicit probability model for heart rate. From this model, easily computed expressions for R-R interval (equation (2)), R-R interval variability (equation (3)), heart rate (equation (5)), and heart rate variability (equation (7)) are derived. These expressions permit real-time estimates of heart rate variability to accompany real-time estimates of heart rate. The tilt table data presented below shows that the heart rate variability estimates provide information that differs from that provided by heart rate estimates. This finding is clinically significant. For example, it is known that fetal distress is associated with changes in heart rate variability before heart rate is affected. Nevertheless, standard fetal monitoring during labor tracks only fetal heart rate.

### EXAMPLE

Protocol

The foregoing estimation methods are illustrated in a tilt-table study of the R-R interval time-series for four healthy subjects. The subjects were two men and two women, ranging in age from 26 to 34 years. The subjects participated in a tilt-table study.

The tilt table is a widely used method of assessing cardiovascular performance in a repeatable and systematic way. By placing the tilt table in a vertical position, a known and constant stress can be applied to the cardiovascular system for known periods of time.

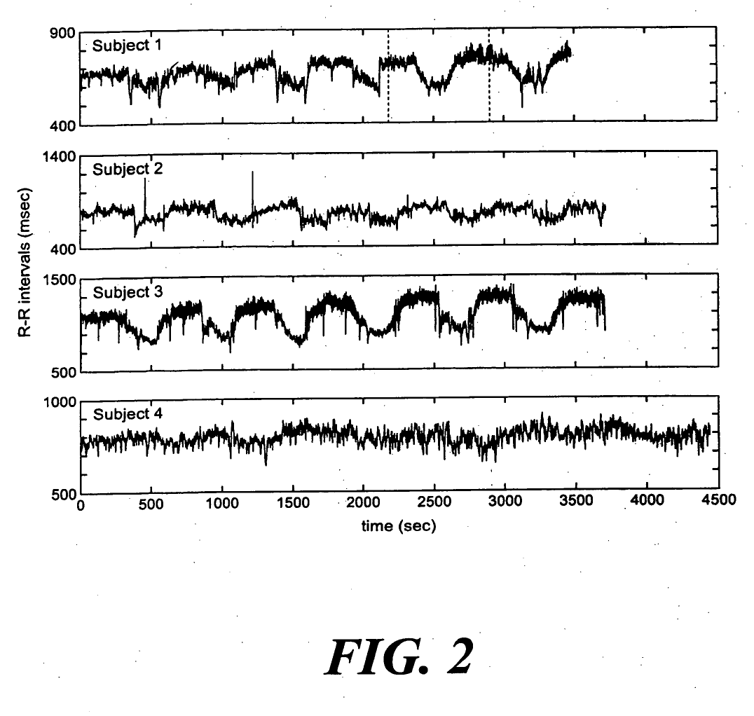

The protocol began with each subject lying supine for five minutes, after which, each subject underwent three types of tilt pairs. The tilt pairs were: rapid tilts, in which the subject moved between a horizontal and a vertical position in less than 3 seconds; slow tilts, in which the subject moved between a horizontal and a vertical position in approximately 1 minute; and stand-ups, in which the subject would stand immediately, supporting his or her own weight, and then lie supine immediately after having been standing. The subject remained in each tilt state for three minutes. Each tilt pair was repeated twice. The order of the tilt pairs was chosen at random for each subject. The procedures lasted from 3,500 to 4,500 seconds. A single lead ECG signal was continuously recorded for each subject during the study, and the R-R intervals were extracted using a curve length-based QRS detection algorithm. The R-R interval series for the four subjects are shown in FIG. 2. The tilt and rest periods are readily apparent in the R-R interval series for the first three subjects. However, they are less evident in the fourth subject. This suggests that each subject mounted a unique response to similar stimuli.

Data Analysis

To choose a model order p and a local likelihood interval l, the HDIG model was fit to the first subject using orders p=2, 4, 6, 8, 10, and 12, with l ranging from 15 to 180 seconds in increments of 15 seconds, and with the shift parameter Δ=5 milliseconds. The AIC and KS plots from this preliminary analysis suggested the use of a model order of either 4 or 6 and a local likelihood interval of 30, 45, or 60 seconds. Each series was then fit to the HDIG model with p=4 and 6, and l=30, 45, and 60 seconds. For comparison, each series was also fit to the renewal inverse Gaussian (RIG) model (p=0) with l=30, 45, and 60 seconds. For further comparison, heart rates were estimated by averaging the R-R intervals over 30, 45, and 60 seconds. This third estimate is referred to as the “local averages” (LA) model. Analysis of all data sets demonstrated that the optimal HDIG model order was p=4, with a local likelihood interval of l=60 seconds. These values were used for all further analyses.

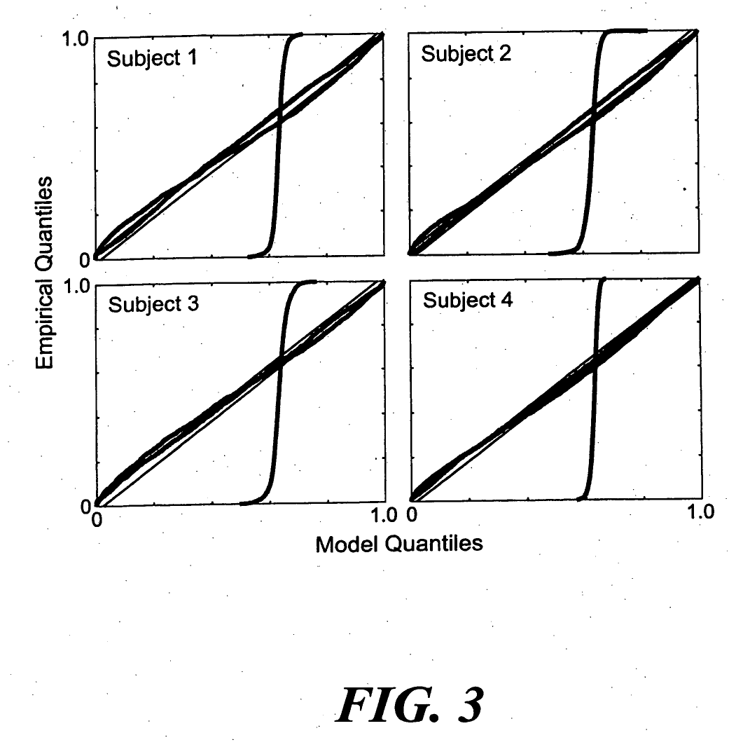

To assess which model best described the data, the KS test based on the time-rescaling theorem was applied to the conditional intensity functions derived from the HDIG (p=4, l=60), RIG (p=0, l=60), and the LA (l=60) models for each of the four data series. As shown in FIG. 3, for all four subjects, the LA model was in poor agreement with the data, as its KS plots deviated appreciably from the parallel 45° lines (thin black lines) that corresponded to the 95% confidence bounds for an exact fit. For all four subjects, the KS plots for the RIG model lay above the 95% confidence bounds for lower quantiles and below the 95% confidence bounds for upper quantiles. In contrast, the KS plots for the HDIG model lay entirely within the 95% confidence bounds for the second subject, and just barely above the upper boundary from the 0.10 to the 0.20 quantile for the third and fourth subjects. The KS plot for the first subject lay slightly outside the upper bound between the 0.20 and 0.80.

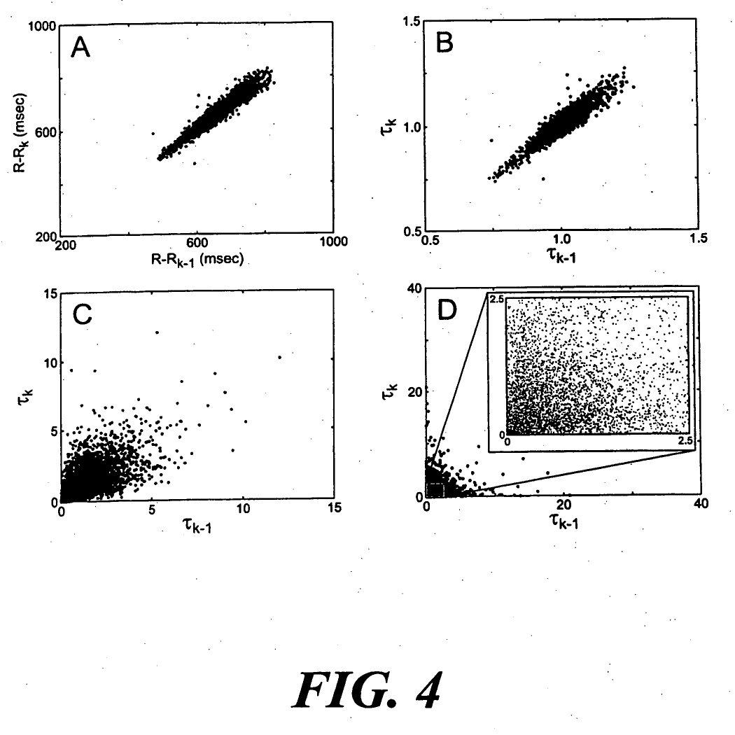

Under the time-rescaling theorem, the R-R intervals are transformed into values of τₖ that should be independent from each other, regardless of the degree of dependence in the original R-R intervals. Therefore, as a further assessment of model goodness-of-fit, τₖ and τₖ₋₁ were plotted against each other for each model. The results of this analysis for the first subject, which were identical to those from the remaining three subjects, are shown in FIG. 4. The τₖ from the model that most closely agreed with the data should have been independent. To the extent that τₖ and τₖ₋₁ were uncorrelated, a scatter plot of one against the other should have resembled a random point cloud. As shown in the scatter plot labeled “A” in FIG. 4, the R-R intervals in the original data were highly correlated, with a correlation coefficient of 0.96. The scatter plot of τₖ against τₖ₋₁ for the LA model, shown in the plot labeled “B” in FIG. 4, featured a persistently high correlation of 0.89. That the LA model's scatter plot closely resembled the original R-R interval plot is not surprising given that this model estimates the conditional intensity function (equation (14)) as a simple, locally constant function. The scatter plot from the RIG model, in FIG. 4, also showed high correlation, with a correlation coefficient of 0.70. In contrast, the scatter plot for the HDIG model, labeled “D” in FIG. 4D was an almost perfect point cloud with a correlation coefficient of only 0.05. These results together with the KS plots from FIG. 3, suggested that, of the three models, it was the HDIG that agreed most closely with all of the four subject's original R-R interval data.

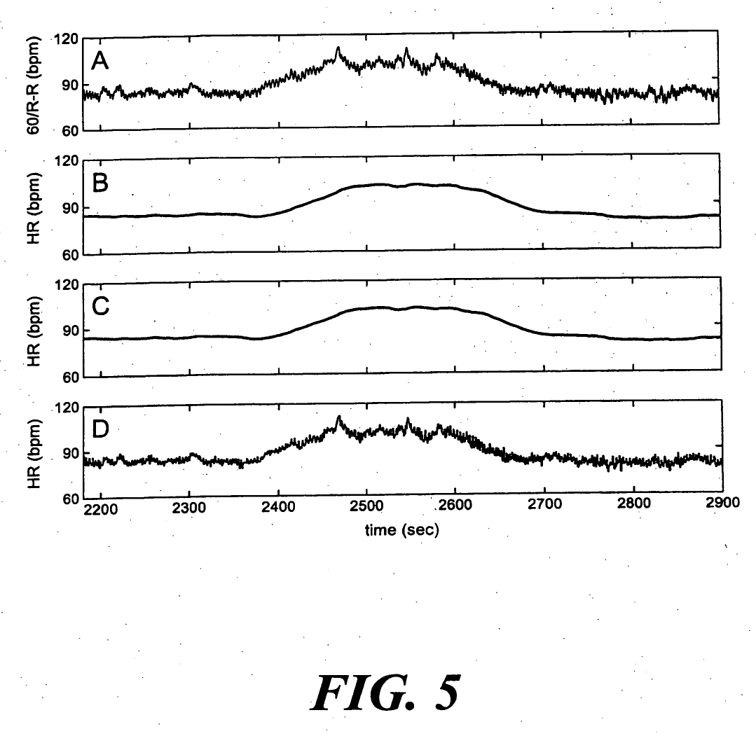

FIG. 5 compares the LA, RIG, and HDIG model instantaneous heart rate estimates for the first subject during the 200 seconds before and after his second slow tilt. The reciprocals of the R-R intervals are shown for baseline comparison in the graph labeled “A” in FIG. 5. During the upward tilt, and while in the vertical position, the subject showed the expected rise in heart rate. During the downward tilt, and with the resumption of the supine position, the subject showed the expected decline in heart rate. In both the baseline and tilt conditions, the estimated heart rate signal for the HDIG model (labeled “D” in FIG. 5) resembled the reciprocal R-R intervals (labeled “A” in FIG. 5) more closely than did the smoother estimates obtained from the RIG model (labeled “C” in FIG. 5) or the LA model (labeled “B” in FIG. 5).

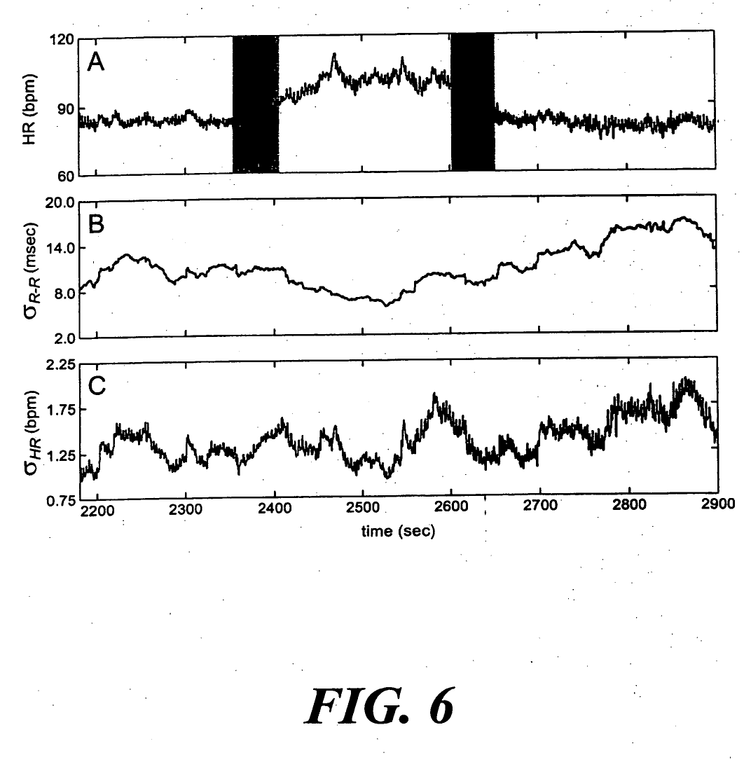

The HDIG model defined by equations (3) and (7) was used to compute instantaneous estimates of both the R-R interval standard deviation (labeled as “B” in FIG. 6) and the heart rate standard deviation (labeled as “C” in FIG. 6) for the first subject. As shown in the graph labeled “A” in FIG. 6, during the pre-tilt rest state, the subject's heart rate fluctuated between 79 and 89 beats per minute (bpm), the R-R interval standard deviation fluctuated between 8.4 and 13.0 milliseconds (as shown in the graph labeled “B” in FIG. 6B), and the heart rate standard deviation fluctuated between 1.0 and 1.6 bpm (as shown in the graph labeled “C” in FIG. 6). During the upward tilt, the subject's heart rate progressively increased from 83 bpm to a maximum of 112 bpm (as showin in the graph labeled “A” in FIG. 6). Paralleling the increase in heart rate, the R-R interval standard deviation decreased, reaching a minimum of 5.7 milliseconds at 2527 seconds (as shown in the graph labeled “B” in FIG. 6). This minimum occurred more than 120 seconds after the upward tilt was completed. The HR standard deviation (shown in the graph labeled “C” in FIG. 6) also decreased during the tilt, however, it did so with more fluctuations. Like the R-R interval standard deviation, the HR standard deviation reached its minimum of 0.94 bpm at 2527 seconds. During the upright state, the subject's heart rate fluctuated around 100 bpm, with short paroxysms of increases and decreases around this value. With the gradual decrease in the tilt angle, the subject's heart rate decreased toward its pre-tilt level of 79 to 89 bpm. A corresponding increase in the R-R interval standard deviation began in advance of the downward tilt and continued for the balance of the rest period, ultimately reaching values of 9.9 to 17.1 milliseconds. The HR standard deviation began its rise in advance of the downward tilt. However, with the decrease in the tilt angle, the HR standard deviation initially decreased. Once the subject was horizontal, the HR standard deviation again increased to a maximum of 2.0 bpm. During the last 60 seconds, there was a rapid decrease in the heart rate standard deviation. Despite having returned to the horizontal position, and although the heart rate after the tilt was nearly the same as it was before the tilt, the dynamics of the R-R interval and heart rate standard deviations upon resuming the rest position suggested that the physiologic state of the subject after the tilt was different from his state before the tilt.

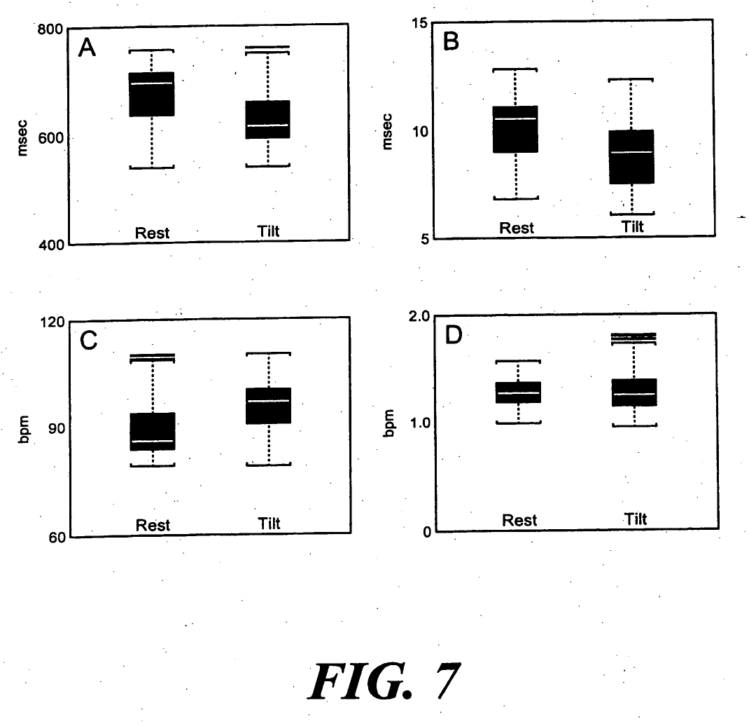

FIG. 7 shows boxplot summaries corresponding to the rest and tilt periods in FIG. 6 of the instantaneous R-R interval mean (as estimated by equation (2)), the heart rate (as estimated by equation (5)), R-R interval standard deviation (as estimated by equation (3)), and the heart rate standard deviation (as estimated by equation (7)). The median (range) of the instantaneous R-R interval mean, as shown in the graph labeled “A” in FIG. 7, was 702 milliseconds (536 to 769 milliseconds) during the rest period and 616 milliseconds (536 to 772 milliseconds) during the tilt period. Similarly, the median (range) of the instantaneous heart rate, as shown in the graph labeled “C” of FIG. 7, was 85.5 bpm (78.0 to 112.0 bpm) during the rest period and 97.4 bpm (77.8 to 112.0 bpm) during the tilt period. Although the medians of the rest and tilt instantaneous mean R-R intervals differ, the ranges of the statistics between the two conditions are the same. The same is true for the median of the rest and tilt instantaneous heart rate between the two conditions. The median (range) of the instantaneous R-R interval standard deviation, shown in the graph labeled “B” in FIG. 7, was 10.5 milliseconds (6.6 to 13.0 milliseconds) during the rest period and 8.9 milliseconds (5.7 to 12.5 milliseconds) during the tilt period. The median (range) of the instantaneous heart rate standard deviation, shown in the graph labeled “D” in FIG. 7, was 1.30 bpm (0.98 to 1.62 bpm) during the rest period and 1.27 bpm (0.94 to 1.88 bpm) during the tilt period. As was true for the instantaneous R-R interval means and heart rate means, although the medians of the instantaneous R-R interval and heart rate standard deviations differ between the rest and the tilt states, the ranges of these statistics are the same in both states. These observations were consistent with the expected tendency of heart rate measures to increase, and heart rate variability measures to decrease, in response to a tilt. However, it is equally apparent that these summary analyses obscure the dynamic behavior of the subject's autonomic nervous system, as revealed by FIG. 6.

The foregoing study of heart rate and heart rate variability began with a statistical model of the human heart beat intervals that incorporated the point process nature of the R-R intervals, the dependence of the interval length on the recent state of autonomic inputs to the SA node, and the dynamic character of these inputs. The history-dependent inverse Gaussian model disclosed herein described the point process structure of R-R intervals and used a local maximum likelihood to estimate the model's time-varying parameters. The inverse Gaussian model described the first passage to threshold of the membrane voltages of the heart's pacemaker cells. Its autoregressive structure described the dependence of the R-R interval lengths on the recent history of the autonomic inputs to the SA node, and its time-varying parameters captured the dynamic character of these SA node inputs. The goodness-of-fit tests showed that a fourth order HDIG model with a sixty-second local maximum likelihood interval gave accurate descriptions of the four data series across multiple transitions between rest and tilt. Most importantly, the tests showed that the HDIG model gave a substantially better fit than either the LA or RIG models. The foregoing analysis provides compelling evidence that, during multiple transitions between rest and extreme physiological conditions, heart rate is accurately described as a stochastic process with non-stationary parameters.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for estimating a statistic associated with a cardiac signal having periodic events, the method comprising:
defining a point process stochastic model of the cardiac signal;
selecting, from a continuum of times, a selected time at which to estimate the statistic;
determining an extent of a censored interval that begins with a most recent one of the events and ends at the selected time; and
on the basis of the point process model and the extent of the censored interval, computing an estimate of the statistic at the selected time.

2. The method of claim 1 further comprising selecting the stochastic model to have a history-dependent inverse Gaussian probability density function.

3. The method of claim 1, further comprising estimating a parameter associated with the stochastic model at least in part on the basis of a history of intervals.

4. The method of claim 3, wherein estimating a parameter comprises computing a maximum likelihood estimate.

5. The method of claim 3, further comprising recursively updating the parameter estimate.

6. The method of claim 1 further comprising selecting the physiologic signal to be a signal derived from fetal heart rate.

7. The method of claim 3, further comprising selecting the number of intervals to be between two and five.

8. A system for estimating a statistic associated with a cardiac signal having periodic events, the system comprising:
a clock for determining an extent of a censored interval beginning at a most recent event and ending at a time selected from a continuum of times; and
a digital filter for implementing a point process stochastic model of the cardiac signal and receiving the censored interval, the filter being configured to estimate the statistic at least in part on the basis of historical data and the censored interval.

9. The system of claim 8, further comprising a signal source for providing the cardiac signal.

10. The system of claim 9, wherein the signal source comprises an electrocardiograph.

11. The system of claim 9, wherein the signal source comprises a fetal heart rate monitor.

12. The system of claim 8, wherein the digital filter is configured to receive the historical data.

13. The system of claim 12, wherein the digital filter is configured to implement a stochastic model having a history dependent inverse Gaussian probability density function.

14. A computer-readable medium having encoded thereon software for estimating a statistic associated with a cardiac signal having periodic events, the software including instructions for causing a computer to:
define a point process stochastic model of the cardiac signal;
select, from a continuum of times, a selected time at which to estimate the statistic;
determine an extent of a censored interval that begins with a most recent one of the events and ends at the selected time; and
on the basis of the point process model and the extent of the censored interval, compute an estimate of the statistic at the selected time.

15. The computer-readable medium of claim 14, wherein the software further comprises instructions for causing a computer to select the stochastic model to have a history-dependent inverse Gaussian probability density function.

16. The computer-readable medium of claim 14, wherein the software further comprises instructions for causing a computer to estimate a parameter associated with the stochastic model at least in part on the basis of a history of intervals.

17. The computer-readable medium of claim 16, wherein the instructions for estimating a parameter comprise instructions for computing a maximum likelihood estimate.

18. The computer-readable medium of claim 16, wherein the software further comprises instructions for causing a computer to recursively update the parameter estimate.

19. The computer-readable medium of claim 14, wherein the software further comprises instructions for causing a computer to select the physiologic signal to be a signal derived from fetal heart rate.

20. The computer-readable medium of claim 16, wherein the software further comprises instructions for causing a computer to select the number of intervals to be between two and five.
